(19) United States
(12) Patent Application Publication
(10) Pub. No.: US 2018/0177743 A1
(43) Pub. Date: Jun. 28, 2018

(54) METHOD OF TREATING PAIN USING RACEMIC MIXTURE OF S-KETAMINE AND R-KETAMINE

(71) Applicant: Gary Jay, Durham, NC (US)

(72) Inventor: Gary Jay, Durham, NC (US)

(21) Appl. No.: 15/849,574

(22) Filed: Dec. 20, 2017

Related U.S. Application Data
(60) Provisional application No. 62/438,449, filed on Dec. 22, 2016.

(51) Int. Cl. A61K 31/135; A61K 47/36; A61K 9/51; A61K 9/00

(52) U.S. Cl. CPC A61K 31/135; A61K 9/0043; A61K 9/5161; A61K 47/36

(57) ABSTRACT

A method of administering a drug formulation comprising intranasally administering a nanoparticle composition of an effective amount of a subanesthetic racemic mixture of ketamine comprising equal amounts of R(−)ketamine and S(+)ketamine, using a chitosan or pectin excipient with an effective amount of pharmacologically acceptable salt thereof.

### CROSS-REFERENCE TO RELATED APPLICATION(S)

Applicant claims benefit of U.S. Provisional Application 62/438,449 filed Dec. 22, 2016 titled “METHOD OF TREATING PAIN USING RACEMIC MIXTURE OF S-KETAMINE AND R-KETAMINE,” incorporated herein in its entirety.

### BACKGROUND OF THE INVENTION

### 1. Field of Invention

The present invention generally relates to pharmaceutical compositions for the treatment of pain, and more particularly relates to pharmaceutical compositions using racemic mixture of ketamine for the treatment of pain such as analgesia, acute and chronic pain, neuropathic, nociceptive pain of all types, acute battlefield pain/injury, opioid sparing and migraine, and cluster headache, post-operative pain, as well as the treatment of depression, PTSD, OCD, battlefield analgesia.

### 2. Description of Related Art

Generally, ketamine is a medication used for anesthesia. Ketamine induces a dissociative state, with certain doses, while providing pain relief, sedation and memory loss. Many are affected by diseases such as psychiatric and neurological diseases, for example, obsessive-compulsive disorder is an anxiety disorder involving obsessions and compulsions. For the treatment of the psychiatric diseases such as depression, schizophrenia, anxiety disorders, and autism spectrum disorder, racemic ketamine appears to be helpful in treating the patient.

Ketamine, in subanesthetic dosages, is one of the drugs that has attracted attention for treatment of patients with Major Depressive Disorder (MDD), bipolar disorder, obsessive-compulsive disorder and Post-traumatic stress disorder (PTSD). PTSD is a prevalent and highly debilitating psychiatric disorder, where the treatment is notoriously difficult. One of the major problems for treating depression is that there are limitations on effects of the antidepressant and effects of its adjuvant therapy. It takes several weeks or more using the antidepressants for the drugs to show their efficiency. In addition, antidepressants are ineffective for treatment-resistant patients. Further, only 50% of patients with depression reach remission. In addition, patients may suffer from various side effects when the dosage of the antidepressant is increased for achieving remission.

Generally, ketamine composition is used for treating psychotic symptoms such as hallucination, delusion and drug dependence. At present, ketamine is used at subanesthetic dosages as an anesthetic and treatment of acute and chronic pain. Intranasal administration of ketamine as a liquid formulation without any excipient makes it difficult to determine exact absorption as well as rates of absorption of the drug composition. Further, it is difficult to determine the pharmacokinetics (PK) of the drug, which requires an additional exacting dosage.

Therefore, there is a need for a novel composition comprising racemic mixture with a pectin or chitosan excipient to treat depression through intranasal (IN) or nose-to-brain (NTB) delivery to treat acute and chronic pain disorders as well as psychological disorders.

### SUMMARY OF THE INVENTION

A method of administering a drug formulation comprising intranasally administering a nanoparticle composition of an effective amount of racemic mixture comprising R(−)ketamine and S(+)ketamine in subanesthetic dosages, using a chitosan or pectin excipient with an effective amount of pharmacologically acceptable salt thereof.

Administering the drug formulation comprising depositing the nanoparticle composition past a nasal valve anatomical feature.

The composition comprises an average particles size smaller than 10 μm in a non-powder formulation.

The composition comprises an average particles size of approximately 200-300 nm in a dry powder formulation.

Treating depression such as PTSD, Autism Spectrum Disorder, acute and chronic pain, post-operative pain, pain secondary to central sensitization, acute and chronic nociceptive and neuropathic pain, migraine and cluster headache, and acute and chronic issues by nature.

Treating acute and chronic nociceptive pain, acute and chronic neuropathic pain, fibromyalgia, migraine, tension-type headache, cluster headache, multiple other forms of pain secondary to cerebral and central hypersensitivity disorders, fibromyalgia, neuropathic, and nociceptive pain

The racemic mixture in a liquid formulation at 10 mg to 80 mg of R(−)ketamine and/or S(+)ketamine.

A ketamine drug formulation comprising a nanoparticle composition of an effective amount of racemic mixture comprising a ketamine using a chitosan or pectin excipient. A nanoparticle size smaller than about 10 μm in a liquid formulation and about 200-300 nm in a dry powder formulation.

Administering the drug formulation past a nasal valve anatomical feature.

Administering the drug by one of a method consisting of intranasal, intravenous, or nose-to-brain (NTB).

Using chitosan encapsulated or dry powder formulations in dosages of 10 mg to 80 mg.

The present disclosure provides a pharmaceutical composition containing racemic ketamine at subanesthetic dosages, via a chitosan nanoparticle for intranasal administration and method of using the said composition for the treatment of stress, acute and chronic pain disorders. Also provided herein is a pharmaceutical acceptable carrier, excipient or diluents comprising an effective subanesthetic dosage of the racemic mixture of equal amounts of R(−)ketamine and S(+)ketamine and an effective amount of pharmacologically acceptable excipient such as a pharmacologically acceptable salt. In some aspects, the pharmaceutical composition is administered by any one of a method consisting of intranasal, intravenous, or nose-to-brain (NTB). In some aspects, the pharmaceutical composition of racemic (S-ketamine and R-ketamine) may be used for treating a variety of ailments. Treatment can be provided to alleviate pain from many causes, acute and chronic, including but not limited to shock; limb amputation (and phantom limb pain); severe chemical or thermal bum injury and the pain of wound repair; sprains, ligament tears, bone fractures, wounds and other soft tissue injuries; dental surgery, procedures and multiple other diatheses which produce pain; reducing labor and delivery pain (ketamine in low doses is not known to have significant adverse effects on the fetus); to help enable painful physical therapy; post-operative pain; radiation poisoning; cancer; acquired immunodeficiency syndrome (AIDS) (neuropathic pain); epidural (or peridural) fibrosis; failed back surgery and failed laminectomy; post laminectomy pain syndrome; sciatica; painful sickle cell crisis; pain secondary to pancreatitis; renal stone induced colic; arthritis; autoimmune disease; intractable bladder pain; migraine headache and others, and more nociceptive, neuropathic, inflammatory and cancer (mixed) pain forms. Psychological conditions can also be treated such as depression, Post-traumatic stress disorder (PTSD), and anxiety. Battlefield pain/injury can also be treated avoiding hemodynamic problems attendant to opioids while getting the soldier to safety and to appropriate medical attention.

In a preferred embodiment, the aqueous formulation of pharmaceutical composition consists of an effective subanesethetic dosage of racemic or S-ketamine and R-ketamine in nanoparticles size. In certain aspects, a method of treating depression includes treating a human individual suffering from PTSD with a therapeutically effective amount of racemic ketamine with a pectin or chitosan excipient. In some aspects, the effective amount of a nanoparticle composition of racemic ketamine with a pectin or chitosan excipient administered via IN (Intranasal) or NTB (nose-to-brain) delivery, the average particles size of the composition is smaller than 10 min a non-powder formulation. In certain aspects, the size of racemic ketamine is 200-300 nm in a dry powder formulation.

In a preferred embodiment, the novel racemic composition in subanesthetic dosages provides both rapid and long-lasting antidepressant effects on diseases exhibiting depressive symptoms, such as depression, bipolar disorder, obsessive-compulsive disorder, PTSD and autism spectrum disorder, as well as helping to decrease pain in multiple diatheses.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this

### DETAILED DESCRIPTION

The foregoing, and other features and advantages of the invention, will be apparent from the following, more particular description of the preferred embodiments of the invention, the accompanying drawings, and the claims.

### DETAILED DESCRIPTION OF PREFERRED EMBODIMENTS

Embodiments of the invention are discussed below. However, those skilled in the art will readily appreciate that the detailed description given herein is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawings.

The invention's primarily focus on racemic mixture of ketamine ((2-(2-chlorophenyl)-2-(methylamino)-cyclohexanone) is used as an anesthetic by anesthesiologists, veterinarians and researchers. It is also currently being used in subanesthetic dosages for the off-label treatment of pain and psychiatric disorders when given IV, PO or IN. The pharmaceutical composition comprises nanoparticle composition of racemic ketamine with chitosan or pectin excipients for the treatment of stress disorders. Also provided herein is a pharmaceutical acceptable carrier, excipient or diluents comprising an racemic mixture of effective subanesthetic dosages of R(−)ketamine and S(+)ketamine, and an effective amount of pharmacologically acceptable salt thereof. In some aspects, the pharmaceutical composition is administered by any one of a method consisting of intranasal, intravenous, or nose-to-brain (NTB). In some aspects, the pharmaceutical composition of racemic (S-ketamine and R-ketamine) may be used for treating depression, PTSD, anxiety, N-Methyl-D-Aspartate (NMDA) enhanced pain including neuropathic pain, acute and chronic, acute and chronic nociceptive pain, as well as central sensitivity disorders such as fibromyalgia, irritable bowel syndrome, migraine, cluster headache, tension-type headache and multiple other acute and chronic pain disorders.

In an embodiment, the ketamine present in the said composition is a chiral compound. Ketamine (or sometimes referred to as RS(+/−)-ketamine) containing equal amounts of R(−)ketamine and S(+)ketamine. R(−)ketamine and S(+)ketamine are also called R-isomer and S-isomer of ketamine, respectively. S(+)ketamine has approximately four times greater affinity. Further, S(+)ketamine has an approximately three to four times anesthetic effect as compared to R-isomer and has greater psychotomimetic side effects. The racemic mixture of S(+)ketamine and RS(+/−)ketamine can serve as a promising and safe antidepressant as well as a non-opioid analgesic as compared to others.

In a preferred embodiment, the method further comprises an intranasal administration of racemic mixture of S-ketamine and R-ketamine to achieve sedation for any surgery. The racemic mixture of S-ketamine and R-ketamine has also been known to have analgesic properties to treat analgesia with sub anesthetic doses of S-ketamine and R-ketamine. The drug is administered by various routes, including intranasal (i.n. or IN), and nose-to-brain (NTB) or other forms such as intravenous (i.v. or IV), intramuscular (i.m. or IM), caudal, intrathecal, and subcutaneous (s.c.).

In an embodiment, the present invention also discloses a method and composition for treating acute and chronic pain and headache, and stress disorders using the racemic mixture of S-ketamine and R-ketamine. The present invention also encompasses methods and compositions for treating PTSD using racemic mixture of ketamine delivered along with a chitosan or pectin excipient. The racemic ketamine is formulated into nanoparticles. The average particle size of the composition is smaller than 10 μm in a non-powder formulation. In certain aspects, the size of racemic ketamine is 200-300 nm in a dry powder formulation. The treatment using the said composition disclosed herein may be administered alone, or supplemented with other antidepressant therapies.

In an embodiment, the racemic S-ketamine and R-ketamine drug may be formulated with the chitosan enhanced racemic ketamine in subanesthetic dosages in nanoparticles and the drug is delivered either intranasally (IN) alone or nose-to-brain (NTB) such as a powder or liquid formulation, with specific devices for IN or NTB. Racemic ketamine is an inexpensive, readily available drug, with minor adverse side effects. Thus, the invention contemplates additional savings to the overburdened health care system. Intranasal administration of this agent is rapid, allowing for fast action of the drug, and easily accomplished even by the non-medically trained.

The compositions and formulations described herein may be for administration orally (solid or liquid), parenterally (intramuscular, intraperitoneal, intravenous (IV) or subcutaneously injection), transdermally (either passively or using ionophoresis or electroporation), transmucosally (nasal, intranasal, vaginal, rectal, or sublingual), or inhalation routes of administration, or using bioerodible inserts. The composition for treatment can be formulated in dosage forms appropriate for each route of administration. The most suitable route in any given case will depend on the particular host, and nature and severity of the conditions for which the active ingredient is being administered. The compositions may be conveniently presented in unit dosage form and prepared by any of the methods well known in the art of pharmacy, and using well-known carriers and excipients. Either of liquid and powder intranasal formulations may be used. The composition comprises racemic ketamine may be combined with a dispersing agent, or dispersant, an excipient (chitosan or pectin) and is administered intranasally in an aerosol formulation optimized for intranasal administration or for nose to brain formulation administration.

In the series of studies, using the racemic mixture as a liquid formulation at 30 mg to 50 mg when administrated via the Intranasal (IN) route has shown the ability of IN racemic ketamine to stop acute and/or breakthrough pain. The liquid formulation of racemic ketamine making the total bioavailability possibly different in each patient (especially if a powder is used instead, than as a liquid formulation with different amounts for different patients). Sometimes the patient may accidentally swallow and the racemic mixture would not be absorbed properly or completely). Using nanoparticles made with chitosan enables a better determination of dosing. Chitosan enhanced nanoparticles enable nose to brain transmission of the drug via the Cribriform plate at the top of the nasal sinus region.

When presenting to a patient, the drug, at subanestheric dosages, can be either a powder for NTB transmission or in a liquid formulation. To enhance Intranasal delivery uses the racemic ketamine as a powder. A specific device can be used to deliver the racemic ketamine mixtures through Intranasal (IN) or IN to nose-to-brain (NTB) delivery. The racemic ketamine mixture is a non-opioidergic analgesic and as used would be opioid sparing. Chitosan enhanced racemic ketamine nanoparticles used for pain via a powder or liquid formulation provided either IN or NTB delivery shows enhanced effect. Different devices would be used for Intranasal (IN) than for NTB drug delivery. With the chitosan enhanced nanoparticles, it shows a better and possibly more accurate delivery of the drug both Intranasal (IN) and nose-to-brain (NTB). When the noted subanesthetic dosages of racemic ketamine are used in a nose-to-brain (NTB) formulation it would enable faster analgesia (and so would IN alone). The racemic and s-ketamine work via the N-Methyl-D-Aspartate receptors to treat acute and chronic nociceptive pain, acute and chronic neuropathic pain, fibromyalgia, migraine, tension-type headache, cluster headache and probably multiple other forms of pain secondary to cerebral and central hypersensitivity disorders, fibromyalgia, neuropathic and nociceptive pain. The racemic mixture packaged with a specific device for IN or NTB use, enables better patient ability to utilize the drug.

A number of advantages over the prior art are exhibited by the current invention. First, while Intranasal liquid ketamine has been used (racemic and esketamine) for pain and depression, the problems are simple: a typical spray bottle will typically send the drug formulation only in as far as 2 cms, and this area of the nasal anatomy is not the most appropriate for transmucosal adhesion and absorption. Frequently, when given this way nasally, drugs may leak out of the nose and/or fall down the esophagus into the stomach, which means the drug encounters gastric acid, which may, depending on the drug formulation, hurt the drug or destroy part or more of it, and if absorbed via the stomach it will go through a hepatic first pass effect that will also have the potential of destroying (metabolizing) a high percentage of the drug. Also, when one looks at the amount of drug in the blood, one doesn't know how much came from the IN use and how much came secondary to the GI route.

In the described method herein, the drug used consists of racemic ketamine encapsulated chitosan nanoparticles, which has never been done before, and dry ketamine powder, both of which have much better mucoadhesion and transmucosal absorption than a liquid formulation. Moreover, the same is true of the dry ketamine powder. Also, both formulations would use a different “inhaler” that would drive the formulations deeper into the nose, past the 2 cm in nasal valve, which would enhance the utilization, and one would be able to determine the exact amount of drug that is given and absorbed. The nanoparticles have not been used before for ketamine, and the dry powder either, with neither specifically sent deeper into the nasal cavity, where there is more ability to absorb the drug and provides a good idea of how much drug is being absorbed transmucosally as the liquid “drips”. Also, no one has previously used a specific device to send a non-opioid analgesic drug (nanoparticles or dry powder) beyond the third nasal turbinate and to the cribriform plate which would allow for nose-to-brain (NTB) absorption. NTB has been shown as effective for brain tumors, using chemotherapy, as well as hormonal entities. The method of intranasally administering a dry formulation of ketamine, a non-opioid analgesic, has not been done before and is very important drug option now during the current “Opioid Crisis”.

The currently disclosed innovation uses two intranasal formulations. No previous intranasal administration uses a nanoparticle formulation of a non-opioid analgesic drug, nor a dry powder formulation specifically placed in a more appropriate part of the nasal cavity (past the 2 cm nasal valve), where more efficient transmucosal absorption takes place. By going past the nasal valve, one can be confident that the administered drug is being absorbed only from the intranasal region. As such, this novel administration of a dry formulation of a non-opioid (or opioid) drug via nose-to-brain absorption has been demonstrated and validated. The use of dry ketamine either as formulation of nanoparticle or dry powder has not been used in a nose-to-brain administration.

The disclosed drug option does not use opioids and lacks the danger of an overdose posed by opioid drug treatments. So the use of non-opioid analgesic with ease of self-administration using a nasal application with no danger of addiction, overdose, and death is new and novel; as well as enabling opioid sparing in opioid users, which would help decrease opioid usage in these patients.

Another novel approach is the deposition of the drug formulations deeper in the nose- and/or nose-to-brain. Exiting nasal application of intranasal drugs use “routine” nasal inhalers that work by placing the drug in frontal 2 cms of the nose, prior to the nasal valve. This is what the typical over the counter and in hospital inhalers do.

Another improvement on prior art is using a dry formulation of an analgesic (non-opioid) so that one can get 90+% of the drug absorbed intranasally, rather than absorption via the nose and the GI tract when drugs are used in liquid formulations.

A final improvement is knowing more precisely how much drug will be absorbed as the powder form and the nanoparticles are much more likely to either get to the deeper nose where more and complete transmucosal absorption takes place, or higher in the nasal cavity where nose-to-brain absorption can take place, which would be done with significantly faster effectiveness.

In an embodiment, a therapeutic dosage of 10 mg to 80 mg of R(−)ketamine and/or S(+)ketamine can be used. The dosages of racemic ketamine (or esketamine) used are sub-anesthetic dosages.

In further embodiments, the aerosol formulation may further comprise a benzodiazepine in a concentration such that the metered amount of the aerosol formulation dispersed by the device contains a dose of the benzodiazepine effective to inhibit dysphoria in the nanoparticle mixture or the dried ketamine powder, with dried benzodiazepine.

The amount of ketamine administered to a patient suffering from chronic pain is about 10% to about 20% of the amount used to induce anesthesia. In another specific embodiment, the dose of ketamine is about 0.01 mg per kg of body weight (0.01 mg/kg) to about 1 mg/kg; preferably about 0.05 mg/kg to about 0.7 mg/kg.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A method of administering a drug formulation, comprising:
intranasally administering a nanoparticle composition of an effective subanestheric amount of racemic mixture comprising R(−)ketamine and S(+)ketamine, using a chitosan or pectin excipient with an effective amount of pharmacologically acceptable salt thereof.

2. The method of administering a drug formulation of claim 1, further comprising:
depositing the nanoparticle composition past a nasal valve anatomical feature.

3. The method of administering a drug formulation of claim 1, wherein the composition comprises an average particles size smaller than 10 μm in a non-powder formulation.

4. The method of administering a drug formulation of claim 1, wherein the composition comprises an average particles size of approximately 200-300 nm in a dry powder formulation.

5. The method of administering a drug formulation of claim 1, further comprising treating/alleviating pain from many causes, acute and chronic, including but not limited to shock; limb amputation (and phantom limb pain); severe chemical or thermal bum injury- and the pain of wound repair; sprains, ligament tears, bone fractures, wounds and other soft tissue injuries; dental surgery, procedures and multiple other diatheses which produce pain; reducing labor and delivery pain (ketamine in low doses is not known to have significant adverse effects on the fetus); to help enable painful physical therapy; post-operative pain; radiation poisoning; cancer; acquired immunodeficiency syndrome (AIDS) (neuropathic pain); epidural (or peridural) fibrosis; failed back surgery and failed laminectomy; post laminectomy pain syndrome; sciatica; painful sickle cell crisis; pain secondary to pancreatitis; renal stone induced colic; arthritis; autoimmune disease; intractable bladder pain; migraine headache and others, and more nociceptive, neuropathic, inflammatory and cancer (mixed) pain forms.

6. The method of administering a drug formulation of claim 1, further comprising treating psychological problems including diseases exhibiting depressive symptoms, such as depression, bipolar disorder, obsessive-compulsive disorder, PTSD, and autism spectrum disorder.

7. The method of administering a drug formulation of claim 1, further comprising treating acute and chronic nociceptive pain, acute and chronic neuropathic pain, fibromyalgia, migraine, tension-type headache, cluster headache, multiple other forms of pain secondary to cerebral and central hypersensitivity disorders, fibromyalgia, neuropathic, and nociceptive pain

8. The method of administering a drug formulation of claim 1, wherein the racemic mixture in a liquid formulation at 10 mg to 80 mg of R(−)ketamine and/or S(+)ketamine.

9. The method of administering a drug formulation of claim 1, further comprising an aerosol formulation that may further comprise a benzodiazepine in a concentration such that the metered amount of the aerosol formulation dispersed by the device contains a dose of the benzodiazepine effective to inhibit dysphoria in the nanoparticle mixture or the dried ketamine powder, with dried benzodiazepine.

10. A ketamine drug formulation, comprising:
a nanoparticle composition of an effective amount of racemic mixture comprising a ketamine using a chitosan or pectin excipient; and
a nanoparticle size smaller than about 10 μm in a liquid formulation and about 200-300 nm in a dry powder formulation.

11. The ketamine drug formulation of claim 10, further comprising administering the drug formulation intranasally past a nasal valve anatomical feature.

12. The ketamine drug formulation of claim 10, further comprising administering the drug by one of a method consisting of intranasal, intravenous, or nose-to-brain (NTB).

13. The ketamine drug formulation of claim 10, further comprising use of chitosan encapsulated or dry powder formulations in subanesthetic dosages of 10 mg to 80 mg.
